(19) United States
(12) Patent Application Publication
(10) Pub. No.: US 2007/0055255 A1
(43) Pub. Date: Mar. 8, 2007

(54) Bioabsorbable Endosteal Fixation Device and Method of Use

(76) Inventor: Mark Girard Siegel, Cincinnati, OH (US)

Correspondence Address: LAFKAS PATENT LLC, 7811 LAUREL AVENUE, CINCINNATI, OH, 45243, US

(21) Appl. No.: 11/162,073

(22) Filed: Aug. 27, 2005

(51) Int. Cl. A61B 17/58

(52) U.S. Cl. 606/72

(57) ABSTRACT

A device for repairing biological connective tissues to bone includes a hallowed elongated body. The elongated body is introduced into a tunnel created in a bone and is positioned to retain connective tissues in position with the tunnel in the bone. The elongated body may be used in conjunction with other known techniques and devices for repairing damaged soft connective tissues.

### BACKGROUND

The various exemplary embodiments of the present invention relate to a device for surgical fixation in bone. More particularly, the various exemplary embodiments relate to a device for surgically anchoring and positioning a ligament or other soft tissue to bone with a bioabsorbable fixation device.

Typically, graft and prosthetic ligaments are utilized to surgically repair and/or replace one or more ligaments damaged by injury or disease. Surgical procedures to repair and/or replace one or more ligaments generally involve forming a tunnel or hallowed channel in bone, positioning one or more graft or prosthetic ligaments in this tunnel in bone, and anchoring the ends of the one or more ligaments. Various devices have been proposed and utilized to secure such ligaments to bone in the tunnel of bone. Examples of such devices include buttons, staples, expanding cones, unicortical screw posts, and interference screws. When interference screws are used, the screws are inserted into the tunnel of bone to engage the tunnel wall and blocks of bone at the ends of the ligament and, thus, provide an endosteum or endosteal fixation there between.

The knee joint is one of the strongest joints in the body because of the powerful ligaments which bind the femur and tibia together. Although the knee is vulnerable to injury as a result of the incongruence and proximity of its articular surfaces, the knee joint provides impressive stability due to the arrangement and interacting strength of its ligaments, muscles and tendons.

In the most simplistic terms, operation of the human knee resembles the actions of a hinge joint. However, in reality, the knee joint provides complicated mechanical movements and maneuverability far more complex than a simple hinge mechanism in regards to the rotation and gliding motions that may occur at the joint. In addition, the motions of flexing and extending the knee require a very detailed structural configuration to facilitate the associated, refined mechanical movements of the knee joint. The knee joint is even so complex that it has slight rotation inward and outward; obviously more complex than a simple hinge.

Anatomically, the knee joint comprises two discs of protective cartilage called menisci. The menisci partially cover surfaces of the femur and the tibia. The menisci operate to substantially reduce friction and impact loading occurring between the femur and the tibia during movement of the knee. The knee is also partly surrounded by a fibrous capsule lined with a synovial membrane. The synovial membrane secrets a lubricating fluid. Strong ligaments on each side of the knee joint provide support to the joint and limit the side-to-side motion and joint opening of the knee. Bursas, which are fluid filled sacs, are located above and below the patella (kneecap) and behind the knee. The bursas provide a means of cushioning the kneecap upon impact and helping with joint lubrication. In addition, there are quadriceps running along the front of the thigh to straighten the knee, and there are hamstring muscles running along the back of the thigh to bend the knee.

Typically, surgical procedures for ligament replacement and reconstruction involve tissues being grafted from one part of the body (autograft) to the original attachment sites of a torn or dislocated ligament. Once the ligament graft has been transplanted, it is then attached to the natural fixation sites of damaged ligament. For example, the replacement of an anterior cruciate ligament (ACL) may involve transplanting a portion of the patellar tendon to the attachment sites of the original ACL to assist in the reconstruction of the ACL in the knee joint.

The expectations of prior art orthopedic procedures typically relate to reconstructing or replacing natural ligaments so as to enable the recipient to return to his or her full range of activity in as short a period of time as possible. To that end, medical researchers have attempted to duplicate the relative parameters of strength, flexibility, and recovery found in natural ligaments of the body. Unfortunately, many of the prior art methods of reconstructing and replacing damaged ligaments have generally proven inadequate for immediately restoring full strength and stability to the involved joint. Furthermore, there has long been a problem of effectively fastening a ligament to a bone surface for the duration of a ligament's healing process, which process involves the ligament graft growing to an adjoining bone mass to restore mobility to the injured joint of an orthopedic patient.

Early ligament replacement procedures traditionally comprised extensive incisions and openings in the knee to attach a replacement ligament to bone surfaces at the fixation sites of the natural ligament. The ends of a grafted ligament were typically secured to exterior bone surfaces by driving stainless steel staples through or across the ligament and into the adjacent bone mass. The legs of the staples are generally adapted for piercing and penetrating tissue and bone mass, while maintaining a ligament at a specified connection site. Other various types of tissue fastening devices, such as channel clamps, were also designed by those skilled in the art. The channel clamps normally differed from the abovementioned staple arrangement in that the channel clamp fixation devices comprise a plurality of components which do not require clinching in the conventional manner, as when setting a staple into a bone surface.

However, the use of stainless steel staples and other related fixation devices have a number of disadvantages. For example, piercing and puncturing of the ligament by the legs of the staples or other fixation devices may result in serious damage to the cross-fibers of the ligament or tissue. Such damage may cause weakening in the tensile strength of the ligament and result in tearing along the cross-fibers of the ligament under normal physical stress. When puncturing or tearing of cross-fibers occurs, the time required for the ligament to heal increases, which in turn results in a significant extension in the amount of time required to rehabilitate the knee joint before allowing the patient to return to normal daily activities.

In response to the problems associated with maintaining a replacement ligament graft at a fixation site, additional devices and techniques were developed offering means whereby a ligament may be retained within a bone tunnel by an endosteal fixation device, such as, for example, an interference screw. The threads of the interference screw are typically bored into the bone tunnel for recessed engagement with the attached bone and one end of the ligament graft, while maintaining the ligament at a fixation site within the bone tunnel. Unfortunately, puncturing, piercing and possible tearing generally results to the cross-fibers of the ligament when the ligament is in direct engagement with the sharp threads of the interference screw. In addition, the interference screw typically requires a ligament replacement graft to be attached to its original bone.

Other problems exist with current interference screws. In particular, the soft tissues comprising tendons and ligaments does not typically provide a good surface for a screw to “bite.” This reduces the ability to easily advance a screw into a desired tunnel. The soft tissues may also deform and wrap around the screw, or the soft tissues may rotate with the screw's advancement and thus end up in the wrong or undesired location within the tunnel.

During flexion or extension of the ligament, tension loads tend to act against the fixation site of the ligament generally causing strain on the ligament against its fixation site. Under such strain, the facing of the threads of the interference screw generally effect a pinching or piercing of the ligament which may cause tearing or dislocation of the replacement ligament under the stress associated with normal physical activities. Consequently, when a grafted ligament suffers cross-fiber damage due to puncturing, piercing or tearing, the healing period for the ligament dramatically increases, thereby in effect, increasing the rehabilitation time for the patient to recover.

Thus, what is desired is a means for securing soft tissues to bone with a greatly reduced threat of mechanical damage to tissues, while also substantially increasing healing time by using bioabsorbable materials for mechanically securing the soft tissues.

### SUMMARY

The various exemplary embodiments of the present invention include a device for substantially securing biological connective tissue to bone. The device comprises a substantially hallow elongated body having a distal end, a proximal end, an inner surface and an outer surface. The distal end includes a first opening. The proximal end includes a second opening. The outer surface comprises one or more anchors to substantially obviate rotation of the elongated body, and the elongated body is comprised of a bioabsorbable material.

The various exemplary embodiments of the present invention also include a method for endosteal fixation, comprising forming a tunnel into a bone. The tunnel comprises a wall. Biological connective tissue is inserted into the tunnel. Then a substantially hallow elongated body is inserted into the tunnel. The elongated body has a distal end, a proximal end, an inner surface and an outer surface. The distal end is truncated and includes a first opening. The proximal end includes a second opening. The outer surface comprises one or more anchors to substantially obviate rotation of the elongated body, and the elongated body is comprised of a bioabsorbable material. The elongated body is positioned in the tunnel such that the connective tissue is anchored to for proper biological healing and bone growth.

### BRIEF DESCRIPTION OF DRAWINGS

Various exemplary embodiments of the present invention, which will become more apparent as the description proceeds, are described in the following detailed description in conjunction with the accompanying drawing, in which:

FIG. 1 depicts a typical knee joint and semitenondosus and gracilis tendons.

FIG. 2 illustrates an elevation view of an exemplary embodiment of the present device.

FIG. 3 illustrates a top view of an exemplary embodiment of the present device.

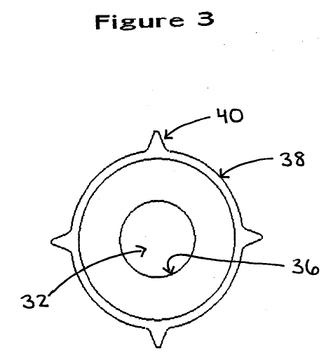

FIG. 4 represents a typical knee joint comprising an exemplary embodiment of the present device anchored in the bone of a femur.

FIG. 5 is an illustration of a bone ready to receive an exemplary embodiment of the present device.

FIG. 6 shows tendons having been anchored and secured to a bone via an exemplary embodiment of the present device.

FIG. 7 is an end elevation view of an exemplary embodiment of the present invention in which tendons are threaded through the present invention and then compressed against the bone by the introduction of a screw against the outer surface of the present device.

FIG. 8 is an end elevation view in which tendons are anchored and secured against the inner surface of the exemplary embodiment of the present device.

FIG. 9 is another end elevation view in which tendons are anchored and secured against the inner surface of an exemplary embodiment of the present device, and then further compressed against the present device by way of a screw.

FIG. 10 is a further end elevation view in which tendons are anchored and secured between bone and the outer surface of an exemplary embodiment of the present invention.

### DETAILED DESCRIPTION

It will be readily understood that the various exemplary embodiments of the present invention, as generally described and illustrated in the Figures herein, could be arranged and designed in a wide variety of different configurations.

These various exemplary embodiments of the invention will be best understood by reference to the Figures, wherein like parts are designated by like numerals throughout.

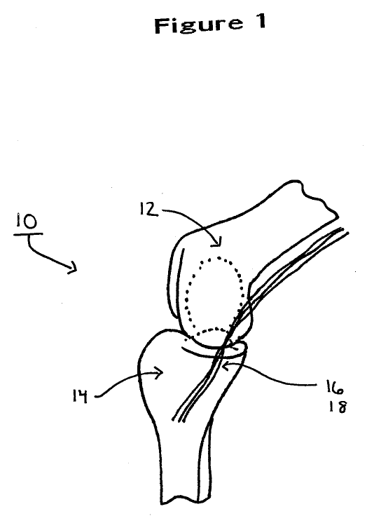

FIG. 1 depicts a typical knee joint 10 comprising a femur 12 and a tibia 14. These bones move by way of mechanical force in the form of muscle contractions transmitted by connective tissues called tendons. Tendons are firmly connected to muscle fibers at one end and to the bone at its other end. A tendon is composed of dense, fibrous connective tissue.

FIG. 1 shows semitendonosus and gracilis tendons 16 and 18 connecting the femur and tibia bones. The semitendonosus and gracilis tendons are often used as grafts for other tendons needing repair.

When grafting and repairing tendons or other connective tissues such as ligaments, the tendons to be repaired need to be securely positioned to limit damage and substantial movement to the repaired tendon. With limited damage and movement to the tendons, better healing can be ensured. Typically, the tendons are secured to adjacent bone.

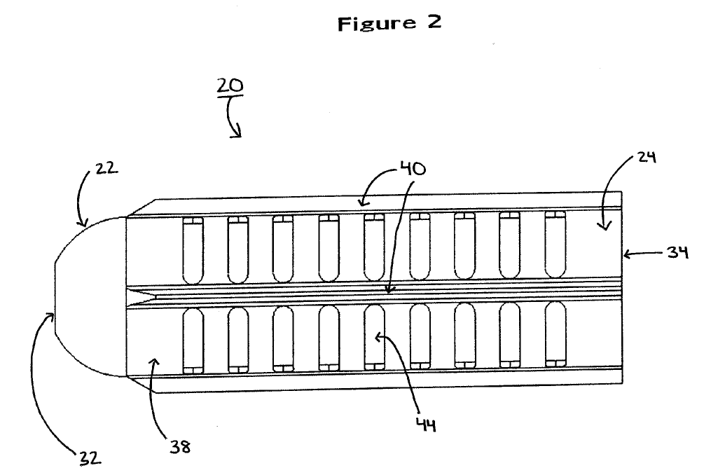

An exemplary embodiment of the present device is depicted in FIG. 2. The exemplary embodiment depicted comprises an elongated body 20 having a distal end 22 and a proximal end 24.

A cross section of the elongated body may be of any geometrical shape such as, for example, a square, rectangle, trapezoid, pentagon, hexagon, circular. However, in a preferred embodiment, the cross section of the elongated body is circular, thus leaving the elongated body cylindrical in shape.

The distal end 22 comprises a first opening 32. The proximal end 24 comprises a second opening 34. As each of the distal end and the proximal end of the elongated body has an opening, the elongated body is hallow. Thus, the elongated body comprises an inner surface 36 and an outer surface 38.

In a preferred embodiment, the distal end of the elongated body is truncated as exemplified in the exemplary embodiment represented in FIG. 2. Truncation of the distal end may allow for potentially easier insertion of the elongated body into a tunnel in bone in order to anchor and secure soft tissue to the bone.

In various exemplary embodiments, the elongated body is preferably comprised of any absorbable or degradable material, including, for example, hydroxyapatite (HA), bone morphogenetic protein (BMP), or any similar substance that encourages bone, tendon, or collagen ingrowth.

In other exemplary embodiments, the elongated body is coated with an absorbable or degradable material, including, for example, hydroxyapatite (HA), bone morphogenetic protein (BMP), or any similar substance that encourages bone, tendon, or collagen ingrowth.

The outer surface of the elongated body may comprise one or more anchors. The one or more anchors are to substantially fix the elongated body when positioned into a tunnel in bone.

In a preferred embodiment, the one or more anchors substantially limit the rotational movement of the elongated body when inserted and positioned within a tunnel in bone.

In the exemplary embodiments wherein the cross-section of the elongated body is of a non-circular geometric shape such as, for example, a triangle, a square, etc., the intersection of the walls comprising the cross-section shape of the elongated body may anchor the elongated body within a tunnel.

The one or more anchors may comprise one or more fin-like projecting radiating outwardly from the outer surface of the elongated body. In the various exemplary embodiments wherein there are multiple anchors on the outer surface, the anchors may be of different lengths and heights. Preferably, in the embodiments in which there are multiple anchors, the anchors are of substantially similar length and height.

In the exemplary embodiments wherein the one or more anchors are fin-like projections, the fin-like projections may be serrated.

The one or more anchors may comprise one or more nodules. The nodules may be substantially linear along the outer surface from the distal end to the proximal end. Or, the nodules may be random or set as a pattern along the outer surface of the elongated body.

In various exemplary embodiments, the elongated body may further comprise one or more slots or series of slots. Such slots preferably provide openings between the outer surface and inner surface of the elongated body.

In surgical procedures to repair damaged ligaments or similar biological tissue, a tunnel in a bone near the damaged ligament is first drilled. The tunnel drilled may be of any size or depth depending on the evasiveness of the surgery, the extent of damage to be repaired, and the surgeon's preference. As the present device is to be inserted and positioned within such a tunnel in bone, the present device is sized for various precise procedures and instances. It is preferred, however, that the present device be sized to be inserted and fit snugly within a tunnel drilled in bone. That is, once inserted and positioned within the bone, the present device should be substantially locked in position within the tunnel of bone.

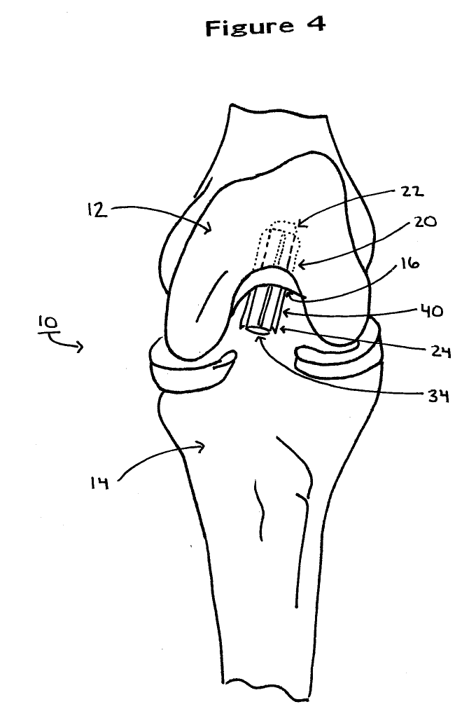

FIG. 4 represents an exemplary embodiment of the present invention partially inserted and positioned within a tunnel 16 drilled in the femur of a knee 10. The distal end 22 is preferably inserted first into the tunnel.

Typically, in endosteal fixation procedures, one end of the ligament graft 50 is secured in the bone. In the case of the drawings, the bone shown is a femur 12, and the grafted ligament is secured to the femoral socket. However, the present device and method can be translated to use in any bones.

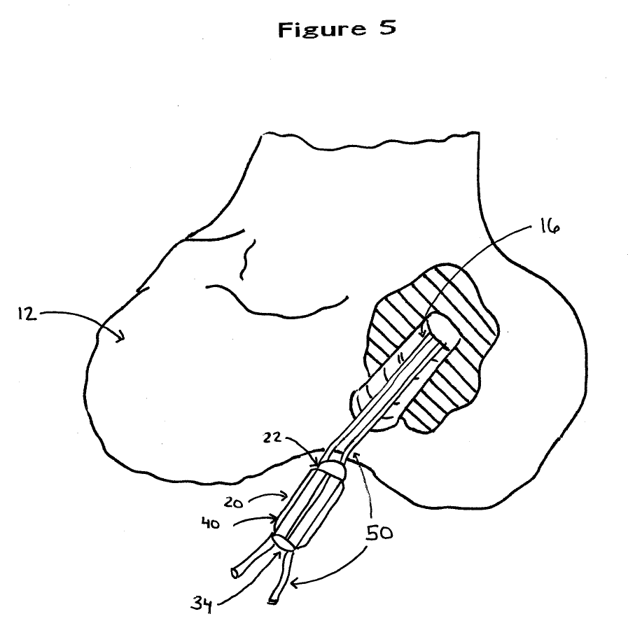

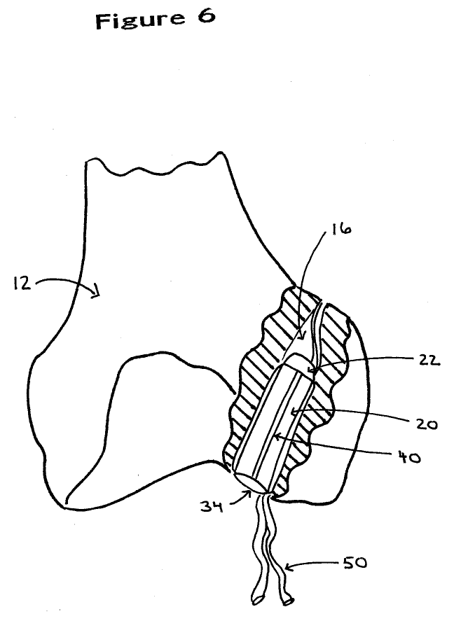

Once one end of the grafted ligament 50 is secured, the other end of the one or more grafted ligaments are pulled taut through the tunnel as shown in FIG. 5. One may use thread or string to attach the one end secured to the bone and then draw the other end through the tunnel. Then, the one or more grafted ligaments are secured within the tunnel drilled in the bone by insertion of the present elongated body device into the tunnel as shown in FIG. 6.

An alternative to the above description may comprise inserting the biological connective tissue through the first opening and second opening of the elongated body. One or more sutures, strings, or a combination thereof, may be connected to the biological connective tissue and pulled through the first opening. Then the biological connective tissue connected to the one or more sutures and the elongated body may be inserted substantially at the same time into the tunnel in the bone. This allows for passing a suture through the second opening at the proximal end.

The grafted ligaments may be secured via the elongated body in any of a combination of techniques.

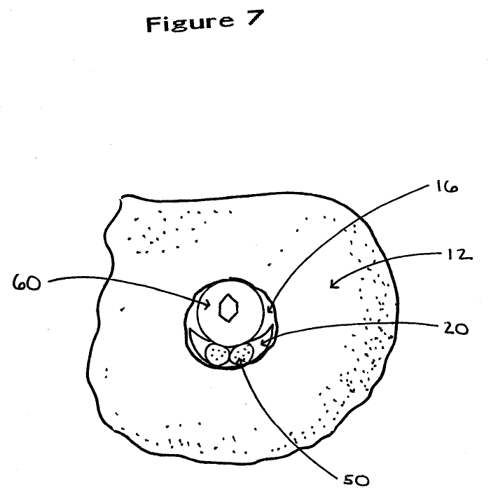

For example, FIG. 7 is an end elevation view of a bone into which a tunnel has been drilled and the elongated body according to the various exemplary embodiments has been inserted. The grafted ligaments 50 have been threaded through the first and second opening of the elongated body. Thus, the grafted ligaments are within the inner surface of the elongated body.

A securing means 60 is then inserted into a space between the outer surface of the elongated body and the tunnel of the bone. The securing means is introduced such that it concentrically compresses the elongated body. Compressing the elongated body as such increases the potential for bone ingrowth into the elongated body and substantially increases the stabilization of the grafted ligaments into position within the tunnel of the bone.

The securing means may comprise a screw, a plug, a wedge, or a combination thereof. Preferably, the securing means is comprised of a material acceptable to biological interaction.

In the exemplary embodiments having a securing means, it is preferred that the elongated body comprises one or more slots. The one or more slots encourage insertion of a screw, and may be spaced to correspond with threads of a screw or pitches of a plug used as a securing means.

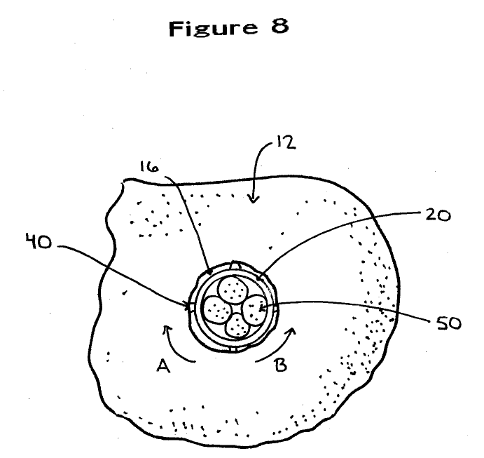

FIG. 8 shows another variation of stabilizing the grafted ligaments with the elongated body according to the various exemplary embodiments of the present invention. As with the variation represented in FIG. 7, the grafted ligaments are fed through the first and second openings of the elongated body such that the grafted ligaments are within the inner surface of the elongated body. The elongated body is then inserted into the tunnel of the bone and is at least slightly compressed such that the grafted ligaments are pushed together and thus are stabilized against the other grafted ligaments and the compressed inner surface of the elongated body.

The elongated body in FIG. 8 includes a set of four fin-like projections serving as anchors. The anchors significantly reduce the rotational movement in direction A and B. Thus, the grafted ligaments are not twisted or torn by rotational movement.

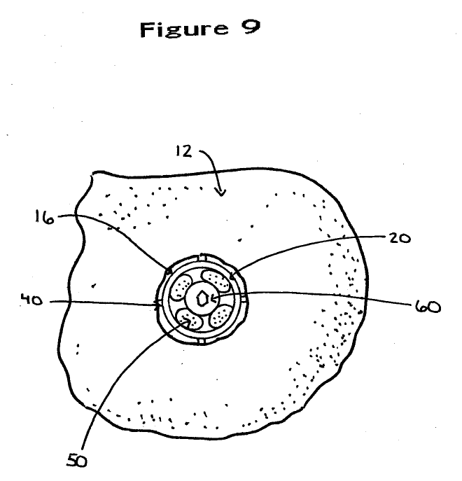

Yet another variation is represented in FIG. 9. This variation of using the elongated body according to the present invention is similar to that shown in FIG. 8, but also includes a securing means being introduced in between the grafted ligaments. The securing means pushes the grafted ligaments out towards the elongated body which is securely compressed against the tunnel of the bone.

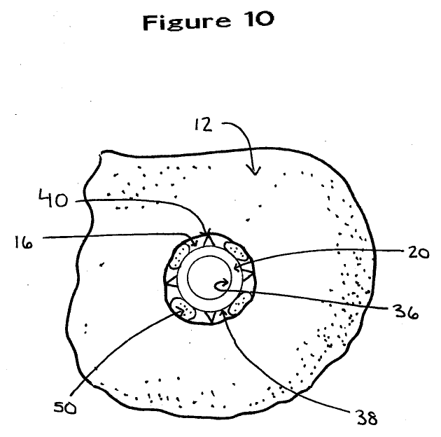

In FIG. 10, the grafted ligaments are directed around the outer surface of the elongated body such that the grafted ligaments are located between the bone and the outer surface of the elongated body. Once again, the elongated body comprises one or more anchors that substantially reduce rotational movement of the elongated body and thus, the grafted ligaments. If desired, or if necessary, a securing means may be introduced between the inner surface of the elongated body to expand the elongated body outward towards the bone and the grafted ligaments.

The present device is advantageous also in that it may be used in conjunction with current techniques and apparatuses for repairing damaged connective tissues.

The above examples are not mean to be exhaustive, nor independent of one another. For example, one or more grafted ligaments may be inserted between the inner surface of the elongated body while other are directed around the outer surface of the elongated body. In either or any above circumstances, the elongated body provided increased stability and positioning of the grafted ligaments, while also promoting growth of the bone.

While this invention has been described in conjunction with the specific embodiments outlined above, it is evident that many alternatives, modifications and variations will be apparent to those skilled in the art. Accordingly, the preferred embodiments of the invention as set forth above are intended to be illustrative, not limiting. Various changes may be made without departing from the spirit and scope of the invention.

What is claimed is:

1. A device for substantially securing biological connective tissue to bone, comprising:
a substantially hallow elongated body having a distal end, a proximal end, an inner surface and an outer surface, wherein the distal end includes a first opening, the proximal end includes a second opening, the outer surface comprises one or more anchors to substantially obviate rotation of the elongated body, and the elongated body is comprised of a bioabsorbable material.

2. The device according to claim 1, wherein the connective tissue is positioned between the outer surface of the elongated body and the bone.

3. The device according to claim 1, wherein the biological connective tissue is positioned between the inner surface of the elongated body and the bone.

4. The device according to claim 1, further comprising a securing means having an outer surface sized to be about the same as the inner surface of the elongated body.

5. The device according to claim 4, wherein the securing means is a screw, plug, wedge, or similar interference fixation apparatus.

6. The device according to claim 1, wherein the one or more anchors on the outer surface of the elongated body comprise one or more fin-shaped protrusions.

7. The device according to claim 6, wherein the one or more fins-shaped protrusions are serrated.

8. The device according to claim 1, wherein the one or more anchors on the outer surface of the elongated body comprise a series of nodules.

9. The device according to claim 1, wherein the elongated body comprises one or more series of slots.

10. The device according to claim 1, wherein the elongated body is comprised of hydroxyapatite (HA), bone morphogenetic protein (BMP), similar material that encourages biological tissue growth, or a combination thereof.

11. The device according to claim 1, wherein the elongated body is coated with hydroxyapatite (HA), bone morphogenetic protein (BMP), similar material that encourages biological tissue growth, or a combination thereof.

12. The device according to claim 1, wherein the distal end is truncated.

13. A method for endosteal fixation, comprising:
forming a tunnel into a bone, wherein the tunnel comprises a wall;
inserting biological connective tissue into the tunnel;
inserting a substantially hallow elongated body into the tunnel, wherein the elongated body has a distal end, a proximal end, an inner surface and an outer surface, such that the distal end includes a first opening, the proximal end includes a second opening, the outer surface comprises one or more anchors to substantially obviate rotation of the elongated body, and the elongated body is comprised of a bioabsorbable material; and
positioning the elongated body in the tunnel such that the connective tissue is anchored to for proper biological healing and bone growth.

14. The method according to claim 13, wherein the biological connective tissue is positioned between the outer surface of the elongated body and the bone.

15. The method according to claim 13, wherein the biological connective tissue is positioned within the inner surface of the elongated body.

16. The method according to claim 13, further comprising driving a securing means into the second opening of the inserted supporting means, wherein the securing means having an outer surface sized to be about the same as the inner surface of the elongated body.

17. The device according to claim 16, wherein the securing means is a screw, plug, wedge, or similar interference fixation apparatus.

18. The device according to claim 13, wherein the one or more anchors on the outer surface of the elongated body comprise one or more fin-shaped protrusions.

19. The method according to claim 13, wherein the one or more anchors on the outer surface of the elongated body comprise a series of nodules.

20. The method according to claim 13, wherein the elongated body comprises one or more series of slots.

21. The method according to claim 13, wherein the elongated body is comprised of hydroxyapatite (HA), bone morphogenetic protein (BMP), similar material that encourages biological tissue growth, or a combination thereof.

22. The method according to claim 13, wherein the elongated body is coated with hydroxyapatite (HA), bone morphogenetic protein (BMP), similar material that encourages biological tissue growth, or a combination thereof.

23. The method according to claim 13, wherein the biological connective tissue is passed through the first opening and the second opening of the elongated body, and the inserting of the biological connective tissue into the tunnel and inserting the elongated body into the tunnel occur at substantially the same time.
